(19) United States
(12) Patent Application Publication
(10) Pub. No.: US 2004/0199093 A1
(43) Pub. Date: Oct. 7, 2004

(54) Therapeutic shoe

(76) Inventor: Anthony Jones, Oak Park, IL (US)

Correspondence Address: Paul L. Brown, Esq., EMRICH & DITHMAR LLC, Suite 2080, 125 South Wacker Drive, Chicago, IL, 60606, US

(21) Appl. No.: 10/826,087

(22) Filed: Apr. 16, 2004

Related U.S. Application Data
(63) Continuation of application No. 10/010,123, filed on Dec. 6, 2001, now abandoned.
(60) Provisional application No. 60/251,899, filed on Dec. 6, 2000.

(51) Int. Cl. A61F 5/00

(52) U.S. Cl. 602/3

(57) ABSTRACT

A shoe for providing therapeutic treatment of a user's foot includes a waterproof flexible shoe member having a sidewall portion and a sole portion adapted to receive a therapeutic solution and a user's foot. The shoe member is structurally arranged to receive and enclose the user's foot and the therapeutic solution therearound. The sole portion of the shoe member includes an upper surface engageable with the user's foot and a lower surface engageable with a supporting surface, with the upper surface having texturizing members thereon which engage the user's foot to treat the foot during movement of the user's foot within the shoe.

### CROSS-REFERENCE TO RELATED PATENT APPLICATIONS

This application claims priority to provisional application No. 60/251,899, filed on Dec. 6, 2000.

### BACKGROUND OF THE INVENTION

In the past, therapeutic devices have been proposed for the treatment of pain relief of feet due to arthritis, joint inflammation, muscle strains and muscle stiffness. Such devices have included soaking-type receptacles containing a therapeutic solution which are adapted to receive a foot therein. However, such devices are bulky, require the use of special therapeutic solutions or hot waxes to treat the patient's foot and prohibit the patient from being mobile during periods of treatment.

When it is desired that such therapeutic devices physically massage a patient's foot, the resultant cumbersome, complex and expensive devices have found only limited acceptance in the marketplace. Also, such prior art devices oftentimes do not provide for the removal of corns or calluses, nor do they treat and ensure that a patient's foot will be fungus free.

Accordingly, it is an object of the present invention to overcome the problems and limitations of such prior art devices.

### SUMMARY OF THE INVENTION

The invention may be incorporated into an improved boot-type shoe device which includes an upper side wall component and a lower sole component structurally arranged for receiving the foot of the wearer therein. The present invention is designed to provide relief to tired feet, after working, running, walking and standing all day, to restore and massage the feet and to facilitate in the removal of corns, calluses and excess dead skin from the foot. With frequent use of the present invention, the present invention significantly reduces and prevents major foot problems by keeping the foot smooth, clean and fungus free. Additionally, the present invention aids and prevents the formation of foot odors.

The therapeutic shoe device in accordance with the present invention provides complete mobility to the user thereof thereby eliminating the requirement that the user must sit, soak and bathe the foot while resting. Additionally, the shoe device enables the user to perform mobile tasks during treatment which massages, scrubs and tones the user's foot to a soft, smooth and healthy condition. Accordingly, the present invention is designed for users who are active and do not always have the time to sit and soak their feet.

Furthermore, the present invention includes texturized structure incorporated therein which engage the foot of the user to massage, scrub and remove excess skin from the foot during treatment. The texturized structure may include brush fibers or molded scrubber-type surfaces which are structurally positioned on the inner surface of the shoe device. Such texturized structure may include a flexible lower sole component having a molded arch support, to permit walking during usage.

The use of treatment solutions together with the therapeutic shoe device aids in the softening of skin and prevents the formation of fungus, foot diseases, and foot odors. The antibacterial or treatment solution may be poured directly into the therapeutic shoe device to contact the foot positioned therein. Unlike foot baths where the soaking liquid or water may be too hot or too cold, the present invention permits the user's own natural body heat to maintain a constant and safe temperature of the treatment solution. The present invention also softens the toenails for safer and easier grooming.

The present invention is, preferably, an insulated, waterproof, flexible boot made of soft, flexible rubber. The inside surface of the boot includes strategically placed brushing or texturing fibers positioned therein and structurally arranged to engage the foot during movement of the foot relative to the flexible boot and to maintain the treatment solution in contact with the foot. Thus, the waterproof flexible boot is, preferably, a non-absorbing waterproof flexible rubber material having built in brush or texturizing fibers that scrub and massage the inserted foot. The waterproof flexible boot may include an exterior covering or be laminated with a impervious nylon material that is waterproof, repels air, aids in temperature control of the treatment solution and provides for attractive appearance and style.

When a foot is inserted within the therapeutic flexible boot, the foot is in contact with and surrounded by the antibacterial solution contained within the soft non-absorbing waterproof rubber lining material. Preferably, a hook and fastener strap positioned at the top of the therapeutic flexible shoe or boot secures the shoe firmly to the lower leg above the ankle. The fastening strap seals the boot around the leg of the user to retain the treatment liquid and the temperature of the treatment liquid at a comfortable level. The sole and arch support aids in support of the boot, and durability. The bottom surface of the sole of the flexible boot may consist of a texturized surface or pattern to provide traction and safety of the invention.

The present invention consists of certain novel features and structural details hereinafter fully described, illustrated in the accompanying drawings, and specifically pointed out in the appended claims, it being understood that various changes in the details may be made without departing from the spirit or sacrificing any of the advantages of the present invention.

### DESCRIPTION OF THE DRAWINGS

The appended claims set forth those novel features believed characteristic of the present invention. However, the invention itself as well as further objects and advantages thereof will best be understood by reference to the following detailed description of a preferred embodiment taken in conjunction with the accompanying drawings, where like reference numerals identify like elements throughout the various drawings, in which:

FIG. 1 is a perspective view of the therapeutic shoe device in accordance with the present invention;

FIG. 2 is a cross sectional view illustrating one embodiment of the therapeutic shoe device in accordance with the present invention;

FIG. 3 is a cross-sectional view illustrating another embodiment of the therapeutic shoe device in accordance with the present invention;

FIG. 4 is a sectional view taken along lines 4-4 in FIG. 2;

FIG. 5 is a schematic view illustrating the insertion of a foot into the therapeutic shoe device in accordance with the present invention;

FIG. 6 is a top plan view illustrating the insertion of the lower leg into the therapeutic shoe device in accordance with the present invention;

FIG. 7 is a top plan view illustrating the closure of the top of the therapeutic shoe device about the lower leg of the user utilizing a strap and hook and loop fastener means in accordance with the present invention; and

FIG. 8 is a side view of a further embodiment of the fastening means in accordance with the present invention.

### DESCRIPTION OF THE PREFERRED EMBODIMENT

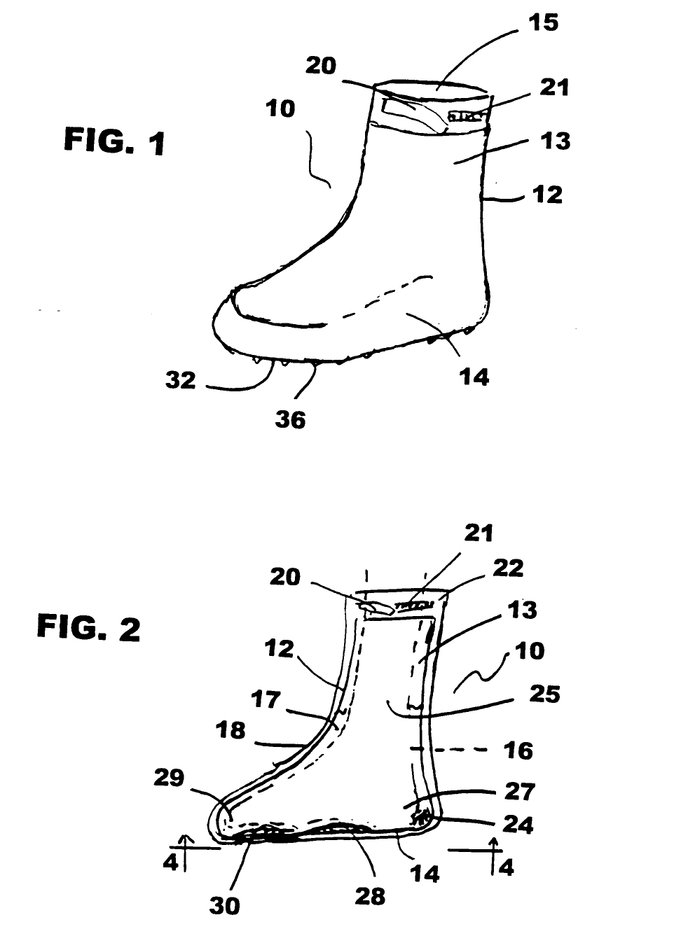

Referring now to the several drawings wherein like numerals have been used throughout the several views to designate the same or similar parts, the drawings illustrate several embodiments of the therapeutic shoe device 10. The shoe device 10 (FIG. 1) is a portable treatment container in the form of a sealed or waterproof boot member 12. The boot member 12 is structurally arranged to receive the foot of a user therein (FIG. 2) and a treatment solution 17 within the sealed boot member. The sealed boot member 12 includes an upper sidewall portion 13 and a lower sole portion 14 and is, preferably, a molded rubber-like flexible member which is waterproof and which is structurally arranged to maintain the treatment solution or fluid within the boot member and in contact with the foot 16 (FIG. 2) of a user. The sealed boot member 12 provides an enclosed bag around the user's foot to keep moisture within contact of the foot and to permit the body heat of the foot to maintain the temperature of the treatment solution within the sealed boot member. The treatment solution may be comprised of an antibacterial soap and water mixture, an antiseptic solution, a peroxide solution or an alcohol solution.

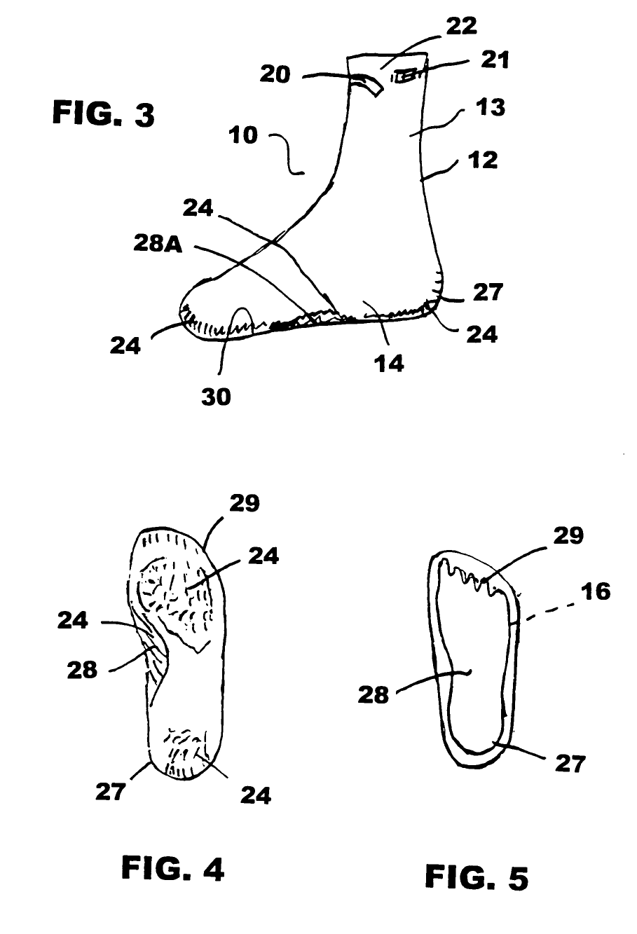

As pointed out above, the sealed boot member 12 is comprised of a molded rubber material which is sufficiently flexible to permit the inside of the boot member to be pulled outwardly or reversed inside-out through the opening 15 (FIG. 1) defined by the upper sidewall portion 13. This reversible feature of the invention permits the user of the therapeutic shoe device to clean the interior of the sealed boot member 12 and to permit removal of any callouses and excess dead skin that has been removed from the user's foot after treatment. As shown in FIGS. 1 and 3, the therapeutic shoe device may be comprised solely of a sealed boot member 12 that includes an interlocking hoop 20, and hook fasteners 21 attached to the end 22 of the upper sidewall portion 13 thereof which permits the upper end of the sidewall portion to be sealed tightly about the ankle 25 of the user, as shown in FIGS. 2, 6 and 7.

The interior of the sealed boot member 12 preferably includes a texturized portion 24 extending from the heel 27 past the arch 28 to the toes 29 of the user when inserted into the boot member 12. The texturized portion is structurally arranged and adapted to engage the inserted foot within the boot, as shown in FIGS. 2 and 5. The texturized portions 24 of the therapeutic shoe device 10 are designed to provide relief to tired feet after working, running, walking and standing, and to restore and massage the feet and to facilitate the removal of corns, callouses and excess dead skin from the foot. The texturized portions 24 are affixed to the upper surface 30 of the lower sole portion 14 (FIG. 3) and may take the form of fibers, brush-like filaments or molded projections that engage the foot to massage and clean the foot during movement of the foot within the therapeutic shoe device 10. Additionally, other portions of the upper surface of the lower sole portion 14 may include an arch support 28a (FIG. 3) and a texturizing pad arranged to engage the bottom of the user's foot.

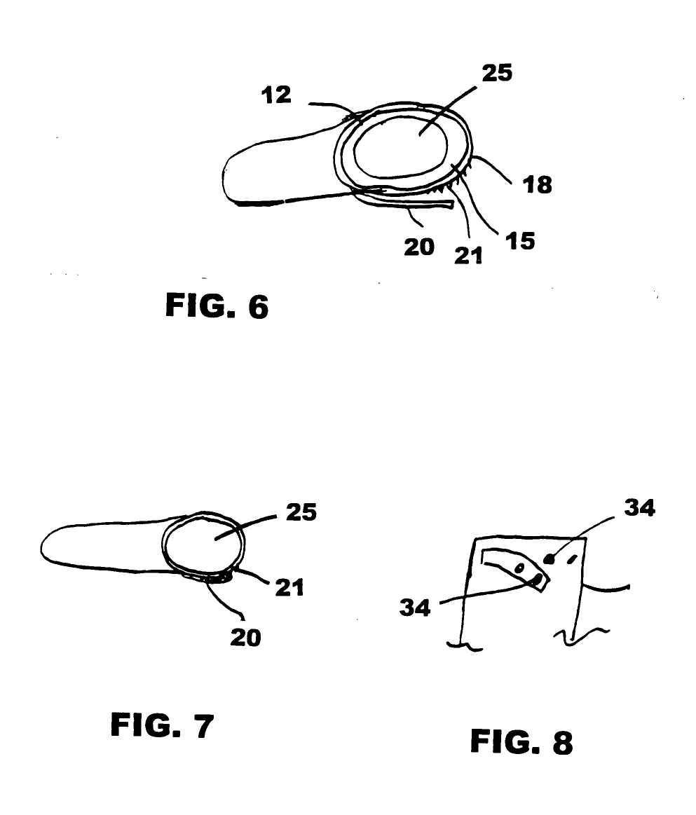

As shown in FIG. 3, the present invention may be limited solely to a sealed boot member 12 that is molded in one piece and which is adapted to receive the foot therein. However, as shown in FIGS. 2 and 6, the therapeutic shoe device 10 may also include a cover member 18 therearound and enclosing the sealed boot member 12. The cover member 18 may be of any particular type material such as nylon and the like, but generally is of the type of material that provides a degree of insulation and protection to the contained sealed boot member 12. As shown in FIGS. 2 and 6, the cover member 18 may include a strap 20 and hook fasteners 21 at the upper edge thereof which cooperate with one another, to permit closure about the ankle/leg 25 of the individual or the user of the therapeutic shoe device 10 to retain the treatment liquid within the sealed boot member 12. As shown in FIGS. 6 and 7, a top view of the therapeutic shoe device illustrates the strap 20 which is mounted to the top of the cover member 18, and the inserted leg/ankle 25 of the user. In FIG. 7, the strap 20 is engaged with the hook and loop fasteners 21 to seal the ankle 25 within the boot member 12. Although not shown in the drawings, the therapeutic shoe device 10 is adapted to receive a liquid, anti-bacterial or treatment solution within the sealed boot member which surrounds the inserted ankle. Sufficient quantities of the treatment liquid are utilized such that when the user inserts a foot therein, the liquid or treatment level substantially engages the foot up to the ankle. Thereafter, the insulating characteristics of the sealed boot member 12 permits the heat generated by the foot of the user to maintain the treatment solution at a temperature substantially the same as the temperature of the user's foot.

The portability of the present invention permits the user of the therapeutic shoe device to walk and move around and do desired tasks while the treatment of the user's foot is being accomplished. This movement by the user of the therapeutic shoe device permits the texturized portions 24 on the upper surface 30 of the sole portion 14 to gently massage, brush and engage the sole, toes and heel of the foot to scrub and remove excess skin during treatment. As pointed out above, the texturized portions 24 may include brush fibers or molded scrubber-type surfaces or projections which are structurally positioned on the upper surface 30 of the sole portion 14 to contact and engage the foot of the user during mobile activities.

The present invention provides that the upper end portion of either the sealed boot member 12 or the upper portion of the cover member 18 may include a securing means such as a strap which is structurally arranged to engage hook and loop fasteners thereon to tighten and hold the shoe device about the ankle of the user. However, it is within the scope of the present invention that the securing means at the upper end of either the boot member or cover member may include overlapping snaps 34 (FIG. 8) which may be engaged to tighten the upper portions of the shoe device about the ankle of the user to secure the therapeutic shoe firmly to the ankle of the user.

Finally, as shown in FIG. 1, the bottom surface 32 of the sole portions 14 of the sealed boot member 12 or the sole portion 14 of the cover member 18 may include a tread or pattern 36 to provide traction and safety for the user during mobile movements of the user when wearing the therapeutic shoe device in accordance with the present invention, as shown in FIG. 1.

1. A shoe for applying a therapeutic solution and treatment for a user's foot, said shoe including a unitary waterproof flexible shoe member having an upper sidewall portion structurally arranged to seal the shoe member about the foot and a sole portion adapted to receive a therapeutic solution and a user's foot therein; said unitary shoe member being structurally arranged to receive and enclose the user's foot with the therapeutic solution therearound, with said sole portion having an inner upper surface engageable with the user's foot and an outer lower surface engageable with a supporting surface, with said inner upper surface of said sole portion having flexible texturizing members thereon which engage the user's foot to facilitate the medical effectiveness of the therapeutic treatment of the foot during movement of the user's foot within the shoe wherein said sidewall portion of the shoe member has an upper end portion having sealing means which secures the shoe to the ankle/leg of the user to prevent escape of the therapeutic solution from the shoe during therapeutic treatment.

2. The shoe in accordance with claim 1, wherein said flexible texturizing members are elongated fibers secured to said inner upper surface of said sole portion.

3. The shoe in accordance with claim 1, wherein said texturizing members are positioned to said inner upper surface of said sole portion to cooperate and engage at least the heel of the user's foot.

4. The shoe in accordance with claim 1, wherein said texturizing members are positioned to said inner upper surface of said sole portion to cooperate and to engage at least the sole of the user's foot.

5 (Canceled).

6. The shoe in accordance with claim 1, wherein said sealing means includes interlocking hoop and loop members.

7. The shoe in accordance with claim 1, wherein said sealing means include at least one snap member.

8. The shoe in accordance with claim 1, wherein said lower surface of said sole portion includes traction members thereon to provide protection of the flexible shoe during usage and to provide engagement with the supporting surface.

9. A shoe for providing the therapeutic treatment of a user's foot, said shoe including
an inner waterproof unitary flexible shoe member having a sidewall portion structurally arranged to seal the shoe member about the foot and a sole portion adapted to receive a therapeutic solution therein, said inner shoe member being structurally arranged to receive and enclose the user's foot with the therapeutic solution positioned therearound, with said inner shoe member having an inner upper surface engageable with the user's foot, with said inner upper surface of said sole portion having flexible texturizing members thereon which engage the user's foot to facilitate the medical effectiveness of the therapeutic treatment of the foot during movement of the user's foot within the shoe; and
an outer cover member enclosing said inner waterproof shoe member and having a sidewall portion and a sole portion having an outer lower surface engageable with a supporting surface to permit movement of the shoe during treatment and protect the inner shoe during the therapeutic treatment of the foot.

10. The shoe in accordance with claim 9, wherein said flexible texturizing members are elongated fibers secured to said inner upper surface of said sole portion of said inner shoe member.

11. The shoe in accordance with claim 9, wherein said texturizing members are positioned to said inner upper surface of said sole portion of said inner shoe member to cooperate and to engage at least the heel of the user's foot.

12. The shoe in accordance with claim 9, wherein said texturizing members are positioned to said inner upper surface of said sole portion of said inner shoe member to cooperate and engage at least the sole of the user's foot.

13. The shoe in accordance with claim 9, wherein said sidewall portion of said outer cover member has an upper end portion having sealing means which secures the shoe to the ankle/leg of the user to prevent escape of the therapeutic solution from the shoe during therapeutic treatment.

14. The shoe in accordance with claim 13, wherein said sealing means includes interlocking hoop and loop members.

15. The shoe in accordance with claim 13, wherein said sealing means includes at least one snap member.

16. The shoe in accordance with claim 9, wherein said lower surface of said sole portion of said outer cover member includes traction members thereon to provide protection of the flexible shoe during usage and to provide engagement with the supporting surface.
